(19) United States
(12) Patent Application Publication
(10) Pub. No.: US 2026/0317469 A1
(43) Pub. Date: Oct. 15, 2026

(54) Population-based Heteropolymer Design to Mimic Protein Mixtures in Biological Fluids

(71) Applicant: The Regents of the University of California, Oakland, CA (US)

(72) Inventors: Ting Xu, Berkeley, CA (US); Zhiyuan Ruan, Berkeley, CA (US); Haiyan Huang, Berkeley, CA (US); Shuni Li, Berkeley, CA (US)

(73) Assignee: The Regents of the University of California, Oakland, CA (US)

(21) Appl. No.: 19/213,862

(22) Filed: May 20, 2025

Related U.S. Application Data
(63) Continuation of application No. PCT/US23/81387, filed on Nov. 28, 2023.
(60) Provisional application No. 63/385,396, filed on Nov. 29, 2022.

(51) Int. Cl. C08F 293/00; G16B 40/30; G16C 60/00

(52) U.S. Cl. CPC C08F 293/005; G16B 40/30; G16C 60/00; C08F 2438/03

(57) ABSTRACT

A plurality of proteins are compared to a plurality of random heteropolymers (RHPs) at a segment level, identifying at least two desired characteristics, and synthesizing an RHP based on the at least two desired characteristics. The synthesized RHP composition includes at least two monomers selected from methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA), or oligo (ethylene glycol) methacrylate (OEGMA).

### CROSS REFERENCE TO RELATED APPLICATIONS

This application is a continuation of PCT/US23/81387, filed Nov. 28, 2023, which claims priority to U.S. Provisional Patent Application Ser. No. 63/385,396, filed Nov. 29, 2022, which are herein incorporated by reference in their entirety.

### GOVERNMENT SUPPORT CLAUSE

The invention was made with government support under grant Numbers HDTRA1-19-1-0011 awarded by the Department of Defense (DOD) Defense Threat Reduction Agency (DTRA), W911NF-21-1-0128 awarded by the Army Research Office (ARO), and 2104443 awarded by the National Science Foundation (NSF). The government has certain rights in the invention.

### FIELD OF THE INVENTION

The present disclosure relates generally to compositions and, more particularly, to a method to design random heteropolymers and compositions thereof.

### BACKGROUND

Biological fluids, the most complex blends, have compositions that constantly vary and cannot be molecularly defined. Despite these uncertainties, proteins fluctuate, fold, function, and evolve as programmed. It is hypothesized that in addition to the known monomeric sequence requirements, protein sequences encode multi-pair interactions at the segmental level to navigate random encounters; synthetic heteropolymers capable of emulating such interactions can replicate how proteins behave in biological fluids individually and collectively. Here, the chemical characteristics and sequential arrangement are extracted along a protein chain at the segmental level from natural protein libraries and used the information to design heteropolymer ensembles as mixtures of disordered, partially folded, and folded proteins. For each heteropolymer ensemble, the level of segmental similarity to that of natural proteins determines its ability to replicate multiple functions of biological fluids including assisting protein folding during translation, preserving the viability of fetal bovine serum without refrigeration, enhancing proteins' thermal stability, and behaving as synthetic cytosol under biologically relevant conditions. Molecular studies further translated protein sequence information at the segmental level into intermolecular interactions with a defined range, degree of diversity, and temporal and spatial availability. This framework provides valuable guiding principles to synthetically realize protein properties, engineer bio/abiotic hybrid materials, and ultimately, realize matter-to-life transformations.

### BRIEF DESCRIPTION OF THE DRAWINGS

The teaching of the present disclosure can be readily understood by considering the following detailed description in conjunction with the accompanying drawings, in which:

FIG. 1 illustrates example blocks and 50-mers along a polymer chain;

FIG. 2 illustrates permutated sequences and a projection of the permutated sequences onto a PCA space;

FIG. 3 illustrates an example two dimensional (2-D) sequence of analysis of proteins and heteropolymers;

FIG. 4 illustrates an example DeepRHP model architecture consisting of a classical VAE equipped with an additional feature-based VAE;

FIG. 5 illustrates an example of PCA projections of RHP and protein latent factors;

FIG. 6 illustrates an example flow chart of a method for synthesizing an RHP of the present disclosure; and

FIG. 7 illustrates a model of the diffusion-limited collision rate estimation of the present disclosure.

To facilitate understanding, identical reference numerals have been used, where possible, to designate identical elements that are common to the figures.

### DETAILED DESCRIPTION

The present disclosure provides examples of a population-based heteropolymer design method to mimic protein mixtures in biological fluids and example compositions that are synthesized using the method.

As discussed above, biological fluids, the most complex blends, have compositions that constantly vary and cannot be molecularly defined. Despite these uncertainties, proteins fluctuate, fold, function, and evolve as programmed. It is hypothesized that in addition to the known monomeric sequence requirements, protein sequences encode multi-pair interactions at the segmental level to navigate random encounters; synthetic heteropolymers capable of emulating such interactions can replicate how proteins behave in biological fluids individually and collectively. Here, the chemical characteristics and sequential arrangement are extracted along a protein chain at the segmental level from natural protein libraries and the information is used to design heteropolymer ensembles as mixtures of disordered, partially folded, and folded proteins.

For each heteropolymer ensemble, the level of segmental similarity to that of natural proteins determines its ability to replicate multiple functions of biological fluids including assisting protein folding during translation, preserving the viability of fetal bovine serum without refrigeration, enhancing proteins' thermal stability, and behaving as synthetic cytosol under biologically relevant conditions. Molecular studies further translated protein sequence information at the segmental level into intermolecular interactions with a defined range, degree of diversity, and temporal and spatial availability. This framework provides valuable guiding principles to synthetically realize protein properties, engineer bio/abiotic hybrid materials, and ultimately, realize matter-to-life transformations.

While molecular precision can be readily achieved inside test tubes, biological fluids are diverse, complex and full of uncertainty. Evolutionarily, the selection of the fittest proteins depends on their surroundings. As a result, natural proteins harmoniously coexist with each other and collectively execute complex tasks with exceptional fidelity amid random fluctuations and external perturbations. Heteropolymer ensembles are designed to mimic protein mixtures in biological fluids via transient interactions with neighboring proteins. Information embedded in the sequence space of natural proteins provides the blueprint to design synthetic heteropolymers to achieve predictable interplay with biological components as protein substitutes, to holistically recapitulate collective behaviors in protein mixtures, and to further gain functions of special proteins while maintaining system compatibility.

It is hypothesized that proteins' chemical and sequence characteristics at the segmental level, as opposed to monomeric level, is the key factor governing how they transiently interact with neighboring molecules and the collective behaviors of biological fluids. Experimentally, mimicking the length distribution of blocks containing consecutive residues of the same characteristics in soluble proteins has been proven effective in designing random heteropolymers (RHPs) to stabilize proteins in non-aqueous media as well as to mimic channel proteins to transport protons. In both cases, the RHP chains have significantly reduced conformational freedom, being either anchored at polar/non-polar interfaces or spanning a lipid bilayer and the observed function readouts are from a subpopulation within each designed RHP ensemble. Nevertheless, these results validated the importance and value of extracting protein sequence information beyond the monomeric level.

In biological fluids, components often interact via random encounters and the whole population needs to be considered. However, when the block-length-based analysis was extended beyond soluble proteins, the block length distribution broadened. Furthermore, the results on the block length distribution contain no information regarding their sequential arrangement within each protein chain. Yet, the effective hydrophobicity of a given block depends on the chemical characteristics of neighboring ones, which affects the probability of it being surface exposed and its spatial location within a globular RHP chain. At the whole chain level, the sequential arrangement of blocks will affect the overall chain conformation, intra-, and inter-chain interactions.

Here, a two-dimensional informative sequence analysis was developed to parameterize and visualize both the chemical characteristics and sequential arrangement at the segmental level using an autoencoder model, and established design rules to modulate RHPs' similarity to proteins as an ensemble. As a test case, a new library of RHPs was developed as synthetic cytosols capable of modulating RHP-protein and RHP-DNA interactions, and their spatial compartmentalization upon microscopic liquid-liquid phase separation (LLPS).

### 2-D Informative Sequence Analysis

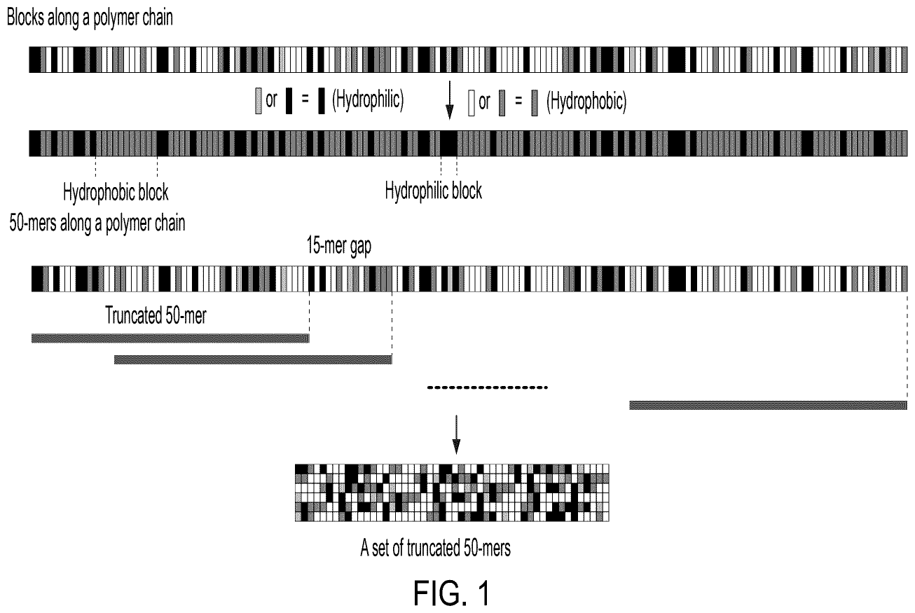

The model was trained on a library of protein sequences including membrane proteins and globular proteins collected from the UniprotKB database. Each sequence was first reduced into four types of pseudo-residues: hydrophilic, hydrophobic, very hydrophobic, and charged, based on the respective side-chain hydrophobicity of amino acids (Table 1, below). Initial sequence analysis was also tested using two or eight pseudo-residues (Tables 2-3). The choice of four pseudo-residues balances the synthetic feasibility of heteropolymers with the accessible diversity and accuracy of segmental interactions. For each protein, the reassigned protein sequence was truncated into a collection of 50-mer segments for analysis, as shown in FIG. 1. The 50-residue interval was chosen because it captured most short-range and long-range residue-residue contacts in protein native conformation.

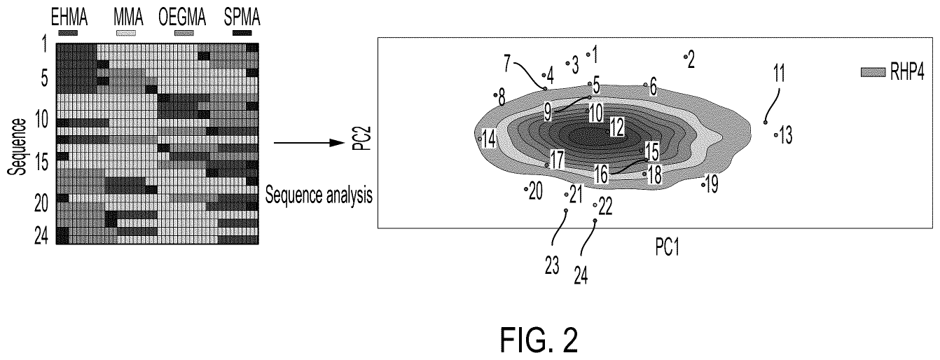

The residue order and local hydrophobicity of each 50-mer were extracted and represented by a low-dimensional vector, which was projected onto a space comprising the first two principal components (PC1 and PC2) from a principal component analysis (PCA). To a first approximation, the PC1 correlates with the apparent hydrophobicity of the 50-mer; the PC2 correlates with the sequential arrangement of different blocks within the 50-mer. A library of hypothetical chains containing the same four blocks arranged in different orders was analyzed for illustration. They are identical based on the block length distribution but can be clearly differentiated based on the 2-D sequence analysis, as shown in FIG. 2. The PC1's dependence on the PC2 further highlights the importance of considering the block sequence along a chain and its potential to modulate inter-chain interactions.

TABLE 1
The conversion from amino acids to RHP monomers (N = 4)

| | | | Monomer |
|---|---|---|---|
| ID | Hydrophobicity | Amino acid | equivalent |
| 1 | Hydrophobic | Cys, Tyr, Ala, Thr, and Gly | MMA |
| 2 | Hydrophilic | Ser, Gln, His, Asn, and Pro | OEGMA |
| 3 | Very | Leu, Ile, Phe, Trp, Val, and | EHMA |
| | hydrophobic | Met | |
| 4 | Charged | Glu, Asp, Arg, and Lys | SPMA or |
| | | | DMAEMA |

TABLE 2
The conversion from amino acids to RHP monomers (N = 2)

| | | | Monomer |
|---|---|---|---|
| ID | Hydrophobicity | Amino acid | equivalent |
| 1 | Hydrophobic | Leu, Ile, Phe, Trp, Val, Met, | EHMA |
| | | Cys, Tyr, Ala, Thr, and Gly | |
| 2 | Hydrophilic | Ser, Gln, His, Asn, Pro, Glu, | OEGMA |
| | | Asp, Arg, and Lys | |

TABLE 3
The conversion from amino acids to RHP monomers (N = 8)

| | | | Monomer |
|---|---|---|---|
| ID | Hydrophobicity | Amino acid | equivalent |
| 1 | Very hydrophobic 2 | Leu, Trp, Ille, Phe | EHMA |
| 2 | Very hydrophobic 1 | Met and Val | HMA^{a} |
| 3 | Hydrophobic 2 | Tyr, Cys, Ala | BMA^{b} |
| 4 | Hydrophobic 1 | Thr, Gly | MMA |
| 5 | Hydrophilic 2 | His, Ser and Gln | OEGMA (Mn = |
| | | | 300 Da) |
| 6 | Hydrophilic 1 | Asn and Pro | OEGMA (Mn = |
| | | | 500 Da) |
| 7 | Positively Charged | Arg and Lys | DMAEMA |
| 8 | Negatively Charged | Glu and Asp | SPMA |

^{a}Hexyl methacrylate.
^{b}Butyl methacrylate.

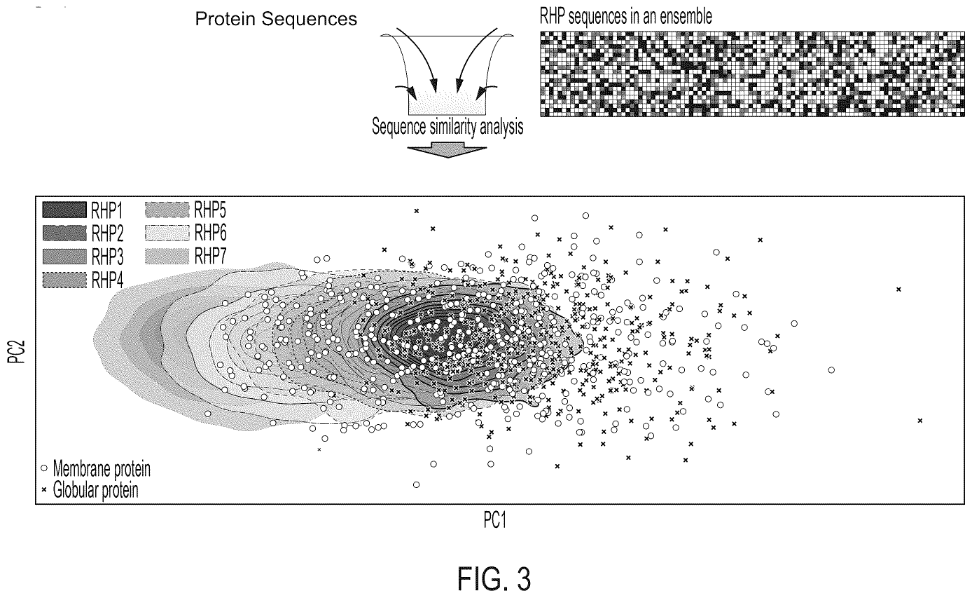

The PCA analysis results for known proteins are shown in FIG. 3 and effectively captures key sequence characteristics for both protein families. Membrane proteins have a sub-population shifted along the PC1 axis compared to that of globular proteins. Thus, the model captures the distinction between the two protein families through their 50-mer segmental hydrophobicity. The large spatial overlap between the two protein families reflects that common protein motifs are present in both protein families. The two protein families were indistinguishable along the PC2 axis and both are more concentrated in the center region of PC2. It is reasonable to speculate that proteins with alternating short hydrophobic and hydrophilic segments are less prone to aggregation and are more preferable through evolution.

### Design and Synthesis of RHP Ensembles

Individual heteropolymers cannot fully capture the protein sequence space and the exact composition of any bio-fluids remains elusive. Thus, a population-based design is required. Individual RHP chains are statistically sequence-defined. They have different monomeric sequences but are statistically similar at the segment level, making them an ideal platform to materialize the segmental information extracted from primary protein sequences. A library of RHP ensembles and two diblock heteropolymers (DHP) were designed and synthesized to match the pseudo-residues used in the initial protein sequence analysis (detailed residue assignments are listed in Tables 1-3, shown above), using 2-4 out of 6 selected monomers including methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA) and oligo (ethylene glycol) methacrylate (OEGMA) with number-average molecular weight (Mn) of 300 Da or 500 Da, respectively. The MMA:EHMA molar ratio was varied in 5 or 10% increments to modulate the distribution of segmental hydrophobicity. Details of all RHPs studied are shown in Table 4.

TABLE 4
Physical-chemical properties of RHPs

| | MMA | OEGMA(500) | EHMA | SPMA | OEGMA(300) | DMAEMA | Mn^{a} | Mn^{b} | Ð^{b} |
|---|---|---|---|---|---|---|---|---|---|
| RHP1 | 70 | 25 | — | 5 | — | — | 18.4 | 20.7 | 1.4 |
| RHP2 | 65 | 25 | 5 | 5 | — | — | 17.9 | 18.3 | 1.5 |
| RHP3 | 60 | 25 | 10 | 5 | — | — | 18.5 | 18.6 | 1.5 |
| RHP4 | 50 | 25 | 20 | 5 | — | — | 36.3 | 27.3 | 1.3 |
| RHP5 | 40 | 25 | 30 | 5 | — | — | 26.5 | 22 | 1.3 |
| RHP6 | 20 | 25 | 50 | 5 | — | — | 25.6 | 20.8 | 1.3 |
| RHP7 | — | 25 | 70 | 5 | — | — | 31.0 | 24.2 | 1.3 |
| DHP1 | 50 | 25 | 20 | 5 | — | — | 17.3 | 17.9 | 1.1 |
| DHP2 | 50 | 25 | 20 | 5 | — | — | 30.5 | 25.8 | 1.4 |
| RHP8 | 60 | — | 10 | — | 15 | 15 | 15.5 | 10.8 | 1.2 |
| RHP9 | 50 | — | 20 | — | 15 | 15 | 15.5 | 11.8 | 1.2 |
| RHP10 | 20 | — | 50 | — | 15 | 15 | 16.4 | 14.6 | 1.1 |
| RHP11 | — | — | — | — | 10 | 90 | 28.7 | 30.1 | 1.3 |
| RHP12 | 50 | 25 | 20 | — | — | 5 | 22.3 | 27.5 | 1.7 |
| RHP13 | 50 | — | 20 | — | 25 | 5 | 17.3 | 11.0 | 1.2 |
| RHP14 | 70 | — | — | — | 25 | 5 | 16.0 | 11.1 | 1.2 |

^{a}Estimated by ¹H NMR
^{b}Estimated by GPC using DMF as the mobile phase.

For each RHP ensemble, 2,000 simulated RHP sequences based on the Mayo-Lewis equation using experimentally-determined reactivity ratios and monomer compositions were analyzed. This sample size is representative of the ensemble, because the occupied PCA space was observed to converge at ~1,500 chains. The segment distribution from proteins and RHP1-7 was projected onto the same PCA space for similarity comparison, as shown in FIG. 3. The occupied PCA space shifts along the PC1 axis, with some overlapping regions, as the monomer composition varies. RHP ensembles populating the left of the PCA space (lower PC1 value) have more hydrophobic segments than those to the right. The PC2 distribution of RHP ensembles are similar to each other and comparable to those of both protein families. RHP ensembles can be designed to match the segmental diversity of both protein families. There is a sizeable overlap between four RHP ensembles, RHP1-4, with globular proteins; RHP1-7 have some overlap with membrane proteins.

### Probing RHP Conformation Via Single-Chain Optical Tweezers

Each RHP ensemble covers a defined range of segmental hydrophobicity and their sequential arrangement. Single-chain end-to-end pulling and relaxation studies were performed using optical tweezers. RHP4 shows large overlap with both families of proteins in PCA space and was chosen for single molecule studies. Results from the first 30 chains are statistically similar to those from a total of 97 chains, indicating that the measured RHP chains should be representative of the ensemble. The RHP ensemble mimics proteins' structural heterogeneity as commonly seen in biological fluids that contain intrinsically disordered, partially folded, and folded states. The force-extension curves (FECs) of 66 out of 97 chains showed no deviation from standard worm-like chain behavior, typical of non-interacting polymers. Four out of 97 chains showed no discrete unfolding “rip” but a “shoulder” feature in the force range of ~ 5-10 pN, reflecting a process of rapid, quasi-equilibrium unfolding or refolding of structures that are only marginally stable. Fifteen out of 97 chains showed discrete rips and 12 out of 97 chains showed a combination of rips and shoulders, indicating cooperative unfolding of mechanically stable structures. When one of the 9 RHP chains with a combination of rips and shoulders was subjected to a constant external force (passive mode), a dynamic transition between distinct force-extension states was observed. The results indicate that certain RHP subpopulations have reversible folding-unfolding transitions similar to those of proteins. FECs were consistent between multiple pulling and relaxation cycles of individual RHP chains. Three subpopulations comprising 31 out of 97 chains form structures with stability of 29.0±22.3 kcal/mol (mean±s.d.). This value falls well within the energy range associated with reversible conformational changes in proteins (~10-90 kcal/mol). Together, an RHP ensemble that matches the PCA space of protein mixtures has a defined range of segmental characteristics and can mimic the proteins' conformational diversity.

RHP/Protein PCA Space Overlap Determines their Interplay

The overlapping PCA regions between proteins and each RHP ensemble indicates similarity in their segmental chemical characteristics, which defines the range of interactions during random encounters in biological fluids. It is hypothesized that to design RHPs as protein mixture mimics, it is more essential to capture the range of intra- and intermolecular interactions within biological fluids rather than replicating their exact compositions, which remain undefined and fluctuating. Thus, the RHPs' similarity to proteins using two tests was evaluated. First, how each RHP ensemble interacts with membrane proteins (MP) during folding post-translation was probed using a cell-free expression platform. Second, how the presence of RHP ensembles in fetal bovine serum (FBS), the most commonly-used biological fluid, affects FBS's ability to support cell cultures during storage and thermal denaturation was measured.

Three representative MPs, OmpT, Aquaporin Z (AqpZ), and PepTso with a C-terminal fused enhanced green fluorescent protein (eGFP), were selected to cover a broad range in the PCA space and different level of folding success without RHPs during cell-free synthesis (FIG. 2). Experimentally, the RHP solution concentration was set to 0.2 wt %, well below the RHP's critical overlap concentration (>10 wt % for RHPs studied) to minimize crowding effects. The RHP:protein collision rate is ~105 s⁻¹ so that the transient RHP:protein interactions are not diffusion-limited based on the translation rate of 10-20 amino acids/s in E. coli and estimated RHP diffusion rate of ~50 μm²/s. In the presence of RHP1-7, a nearly two-fold increase in the AqpZ-eGFP protein yield was observed using S-methionine labeling, and there were improvements in the AqpZ-eGFP's folding status. Both results are positive indications of RHPs' protein-like behavior. RHP4 has the highest level of overlapping PCA space with proteins and was the best performer. Similar trends were observed for PepTso with RHP5-6 being the best two performers. OmpT has a fairly good folding status without RHPs; this was attributed to its lower apparent hydrophobicity compared to the other MPs, as seen by its higher PC1 value. RHP1-3 with higher PC1 values were the most effective in mediating OmpT folding, whereas RHP 6-7 with lower PC1 values have deleterious effects.

The interplay between RHP ensembles and biological fluids upon thermal denaturation was further probed as an accelerated case of how proteins under stress sample different conformations and interact with surrounding molecules. Studies were carried out using FBS, a complex mixture of over 1,000 proteins and other components. Without RHPs, precipitates formed in the FBS solution within several days when stored at room temperature. When RHPs were added, visual inspection showed a significant reduction in the formation of precipitates over a month without refrigeration. The precipitation was more obvious when the FBS was heated without RHPs for 2.5 hrs at 52° C. (FIG. 2E), indicative of increased protein denaturation. When different RHP ensembles were compared, RHP4 performed better than RHP7 and RHP2 in stabilizing FBS and was used for subsequent studies. When the thermally treated FBS was used as a growth supplement for in-vitro cell culture of NIH3T3 fibroblast cells, there was a 19.1±8.5% (mean±s.d.; n=8) reduction in the cell viability. When 0.5 mg/ml of RHP4 ensemble was added into FBS prior to heating, the cell viability was retained at 93.0±12.2% of control FBS without thermal treatment. The cell viability increased to 95.9±10.8% and 104.8±7.8% at a RHP4 concentrations of 1 mg/ml and 2.5 mg/ml, respectively. FBS is a biological fluid commonly used in cell culture, so these studies suggest that RHP ensembles with matching PCA spaces can interact with proteins as if they were proteins themselves. In both studies, specific factors such as molecular chaperones, osmolytes, and crowding effects for protein stabilization were minimized. These results further confirmed the ability of RHP ensembles to navigate through compositional uncertainties during random encounters.

RHP segment surface availability depends on local RHP-protein interactions

Each RHP ensemble includes an enormous number of segments that span a range of segmental hydrophobicity and sequential arrangements within an RHP chain. Ultimately, the availability of these segments during random encounters governs the apparent protein-RHP interactions. Thus, whether the spatial arrangement of segments can be influenced by their immediate environment and the segmental sequence was probed. Solution small-angle x-ray scattering (SAXS) studies showed that RHP4 in water formed a single-chain nanoparticle, 8.8 nm in average diameter, to bury hydrophobic monomers (FIG. 3). ¹H-NMR studies of RHP4 in D20 showed that the surface-exposed segments are more hydrophilic and mobile; the buried segments are more hydrophobic and have a low tendency to snorkel to the surface of globular RHP chains, consistent with previous atomistic molecular dynamic simulations. However, from 1H-NMR, the full width at half maximum (FWHM) accounting for the end-methyl protons of the EHMA side-chain and backbone methyl protons transitioned from being fairly broad to sharp upon heating from 25° C. to 70° C. In the same temperature range, the FWHM of protons from the end methyl group in the OEGMA side chains remained sharp consistently. This result suggests buried hydrophobic residues/segments become more flexible and solvated upon heating. Adding DMSO into the D20 has a similar effect to heating, and also caused the proton peak of the EHMA side chains to sharpen.

During random encounters, the molecules in contact with an RHP can locally solvate and lower the energy barrier to surface-expose amphiphilic or hydrophobic RHP segments. No method exists to directly image the RHPs' conformations when they transiently interact with proteins, so an atomistic MD simulation was performed. In the simulation, a nonpolar hexane nanobubble was placed near an RHP4 in water. The simulations showed that an RHP4 globule can be fused and subsequently unraveled at the interface very quickly (approx. 100 ns). This is in stark contrast to frozen RHP4 backbones observed in pure water. There is a substantial conformational rearrangement of RHP at the hexane nanobubble/water interface, shielding nonpolar groups from exposure to water. When all studies are considered together, it is reasonable to conclude that an RHP chain can effectively modulate its side-chain distribution based on the proteins it contacts, and can thus provide matching segments on-demand to assist proteins as they traverse back to their native states.

An RHP chain can be viewed as an equivalent freely jointed chain with segments spanning a range of segmental hydrophobicity. The exchange dynamics of each segment depend on its hydrophobicity and length, analogous to the single-chain desorption/exchange kinetics of amphiphilic block copolymers. To understand how the segment length affects RHPs' conformational plasticity, two diblock heteropolymers (DHPs), (PMMA-co-EHMA)-b-(POEGMA-co-SPMA) were synthesized, with the same composition as RHP4, but different molecular weights (Mn (DHP1)=17.3 kDa and Mn (DHP2)=30.5 kDa). DHPs have multimodal distributions in the PCA space that account for 50-mers from the PMMA-co-EHMA block, in-between block, or POEGMA-co-SPMA block, respectively. The occupied PCA space shifts along both PC1 and PC2 axes, leading to a significant deviation from the protein PCA space. In an assay of AqpZ folding status, DHP1 could increase the eGFP fluorescence intensity during translation, but did not perform as well as its RHP counterparts. Negligible enhancement of eGFP fluorescence was detected when DHP2 was used.

DLS measurements showed that both DHPs have bimodal size distributions with a mean diameter of 56.2 nm for DHP1, and 145.5 nm for DHP2, much larger than the 9.1 nm diameter of their RHP counterpart. Thus, the PC2 value is important for predicting the RHPs' tendency to form large assemblies that will compromise the PC1 similarity between RHPs and proteins. The prevalence of short hydrophobic/amphiphilic blocks in RHPs is critical to provide its conformational flexibility, thus ensuring segments' availability.

### Designing New RHP Ensembles as Cytosol Mimics

Biomacromolecules need to function coherently regardless of their own specialties. It is hypothesized that proteins' sequence space encodes how protein mixtures behave beyond individual interactions. Therefore, a population-based design approach for synthetic protein analogs is more meaningful than pursuing molecularly-precise formulations. In comparison to the model blends currently used to replicate biological multi-scale phase behaviors, RHP solutions are more relevant because they access key characteristics such as chemical diversity, composition complexity, and uncertainty. As a test case, a new RHP library was synthesized to mimic cytosols with common functions including: (1) tunable microscopic LLPS under biologically relevant conditions; (2) the ability to interface and modulate protein folding and stability; (3) compartmentalization of biomolecules such as DNAs to modulate their bioavailability. Designing RHPs to fulfill multiple functions, each with a high level of biological importance, will test RHPs' similarities to natural proteins and the robustness of population-based design.

RHP8-14 (Table 4) were synthesized using 2-4 of the available monomers: MMA, EHMA, OEGMA (Mn=300 Da), OEGMA (Mn=500 Da), and DMAEMA. Two OEGMA monomers with different sidechain lengths and DMAEMA were chosen to implement and modulate temperature-dependent intermolecular interactions to access LLPS under biologically-relevant conditions. The RHP monomer ratios were selected to have different levels of overlap in the 2-D PCA space with known proteins undergoing LLPS behaviors (from LLPSDB database). Sequence analysis showed that RHP8-10 overlap with the 2-D PCA space of proteins undergoing LLPS, and RHP11 lies outside of the protein space.

RHP8-10 formed spherical droplets suspended in the buffer at 47° C. for less than one minute. The cloud point temperatures of RHP8, 9, and 10 solutions at 1 mg/ml (sodium phosphate buffer, 50 mM, pH 7.0) were determined to be 43° C., 40° C., and <25° C., respectively. Under the same buffer condition, RHP11 showed no phase separation even up to 70° C. Thus, matching the PCA space between proteins and RHP ensembles is an effective approach to replicate proteins' collective behaviors, such as LLPS.

RHP-protein interactions with and without LLPS using RHP12 and RHP13 were studied. Both have the same monomer ratio as RHP4 except that SPMA was replaced by DMAEMA and the side chain length in OEGMA was varied. RHP12 has a cloud point temperature of ~67° C. and the cell-free AqpZ-eGFP folding assay at 37° C. confirmed that RHP12 (0.2 wt %) can facilitate AqpZ folding. In comparison, RHP13 has a cloud point temperature lower than 25° C., underwent LLPS at 37° C., and showed no effect in the AqpZ-eGFP folding. A control experiment showed that RHP13 did not interfered with eGFP expression or folding post translation. How the formation of liquid droplets might affect proteins upon thermal denaturation using a common enzyme, proteinase K (ProK), was also assessed. RHP1 has the most PCA space overlap with ProK and was the best performer as ProK's thermal protectant. RHP1's monomer ratio was adopted to synthesize RHP14 by replacing SPMA with DMAEMA to induce LLPS. In the presence of RHP14, ProK retained 74±5% enzyme activity after being heated at 62° C. for 10 min. In the control experiments without RHPs, ProK lost ~97% of its enzymatic activity. RHP14 has a cloud point temperature of 33° C. Confocal studies showed that the fluorescein-labeled ProK was excluded from RHP14 liquid droplets.

In comparison, only 25% of ProK activity was retained with RHP1 that showed no LLPS. Together, these results suggested that once the LLPS occurred, the hydrophobic RHP segments and some amphiphilic RHP segments became inaccessible to proteins outside of the droplets. These segments are the key to mediating MP/water interactions and assisting MP folding. For ProK, their absence reduced the probability of misfolding during thermal denaturation. These results also indicated these hydrophobic/amphiphilic segments provided the driving forces to form the liquid droplets. NMR studies showed no difference in the monomer compositions between the RHPs inside of the liquid droplets and the original RHP ensemble. Thus, it is reasonable to speculate that the inter-chain interactions within the droplets are dominated by the apparent segmental hydrophobicity within the RHP chains instead of the whole chain.

RHP10 formed many small droplets which ranged from hundreds of nm to a few μm in size. They diffused, collided with one another, and fused together within a few minutes. Cyanine 3 amine (Cy3) dye was concentrated in the droplets and used to probe the local viscosity within droplets via Fluorescence Recovery after Photobleaching (FRAP). Nearly complete fluorescence recovery was observed with a characteristic recovery time of 385±15 s. This time scale is comparable to that of MEG-3 proteins (128-384 s) in P granules. The RHP droplets have a fluidity similar to membraneless organelles, and offer a promising path to their synthetic mimics.

It is hypothesized that the design rules used here to modulate RHP-protein interactions can also be applied to tailor how RHPs interact with DNA. Fundamentally, RHPs enable us to evaluate energetic competition between a wide range of interactions, going beyond the solely electrostatic interactions commonly studied in coacervation. When 1 μM of 24-mer single stranded DNA (ssDNA, 24 nt) was added to the RHP10 solution (1 mg/ml, sodium phosphate buffer, 50 mM, pH 7.0), ssDNA was selectively encapsulated inside the liquid droplets at 37° C. Similar results were observed for a 24-base pair FRET-pair labeled double stranded DNA (dsDNA). FRET was observed inside the liquid droplets, indicating the absence of dsDNA dissociation. When liquid droplets carrying complementary FRET-pair labeled ssDNA were mixed, these droplets fused. Subsequently, complementary ssDNA strands formed duplexes and emitted FRET over the course of 25 min. These results confirmed that the RHP-DNA interactions are strong enough for selective partition but do not interfere with specific DNA pairing interactions. Thus, designed RHPs can capture the range of intra- and inter-molecular interactions required to be compatible with multiple biological processes occurring inside of cytosol, making them viable building blocks toward bio/abiotic materials.

The present disclosure clearly demonstrates the feasibility of designing heteropolymers as an ensemble to mirror protein mixtures in biological fluids despite the inexact formulations of these complex blends. Proteins' segmental sequence information can guide statistic sequence control in RHPs to holistically replicate protein functions. Being orthogonal to molecular-precision driven design, the population-based approach is actually advantageous to navigate through chemical diversity and unpredictable fluctuations abundant in protein's native environments and ultimately, to interface synthetic materials and biological systems seamlessly.

### Materials

Chemicals were purchased from Sigma Aldrich Chemical Co. or Fisher Scientific International, Inc. unless otherwise noted. Water was purified by a Milli-Q water filtration station (18.2 QM cm) before use. Azobisisobutyronitrile (AIBN) was recrystallized from ethanol before use. Inhibitors were removed using cryodistillation (methyl methacrylate, 2-ethylhexyl methacrylate) or by passing through a short column of neutral alumina (ethylene glycol methyl ether methacrylate (Mn ~500 g/mol). 3-sulfopropyl methacrylate potassium salt (98%) prior to polymerization. Ethyl-2(phenylcarbanothioylthio)-2-phenylacetate, Trioxane (TCI, internal standard for 1H NMR analysis), and Dimethylformamide (DMF, solvent) were used without further purification. Cell-free transcription/translation system (PURExpress®) was purchased from New England BioLabs Inc. Plasmid Aqpz-GFP was a gift from Prof. Daniel L. Minor, Jr. (University of California, San Francisco). Plasmid pWaldoGFPe_PepTSo was a gift from So Iwata & Simon Newstead (Addgene plasmid #58334). pET28-OmpT was a gift from Neil Kelleher (Addgene plasmid #68862). Anti-GFP antibody (B2) was purchased from Santa Cruz Biotechnology. Goat anti-mouse alkaline phosphatase secondary antibody conjugate was purchased from Bio-Rad Laboratories. End-modified oligonucleotides were purchased from IDT Co. Proteinase K was purified by a desalting column before use. Dulbecco's Modified Eagle Medium (DMEM) and cell culture plates were purchased from Corning Inc. The fetal bovine serum (FBS) and phosphate buffered saline (PBS) were purchased from Gibco Inc. The 3-(4,5-dimethylthiazol-2-yl)-2,5-diphenyltetrazolium bromide (MTT) and pancreatin were purchased from Invitrogen Co. The NIH3T3 cell line was provided by UCB Cell Culture Facility.

### Methods—Random Heteropolymer (RHP) Synthesis

The synthesis of RHPs was carried out using reversible addition-fragmentation chain-transfer polymerization (RAFT). Polymerization solutions were prepared by mixing the requisite amounts of reagents and added into 25 ml glass Schlenk. The solutions were subject to 3 freeze-pump-thaw cycles before the Schlenk was placed into the oven. The polymerization solutions were held at 70° C., typically for 4-8 hours. Global monomer conversion was determined by 1H-NMR on crude reaction mixtures in DMSO-de with trioxane as an internal standard. The polymerization solution was then precipitated by dropwise addition to rapidly stirred pentane. The resultant polymer was then dissolved in water and transferred to a 2,000 MWCO dialysis bag and dialyzed against distilled water for three days. The purified polymer was then subject to lyophilization.

Diblock Copolymer p(MMA-r-EHMA)-b-p (OEGMA-r-SPMA) synthesis

An example of the preparation of p (MMA-r-EHMA) 53-b-p (OEGMA-r-SPMA)₂₂(Mn(NMR)=17327 g/mol) is given.

P(MMA-r-EHMA) macro-CTA synthesis MMA (1072.71 mg, 10.71 mmol), 2-EHMA (849.86 mg, 4.29 mmol), CTA (2-Cyano-2-propyl 4-cyanobenzodithioate, 32.13 mg, 0.013 mmol), AIBN (2.14 mg, 0.0013 mmol), DMF (1 ml) were mixed in a 20 ml glass vial. The reaction mixture was deoxygenated by N₂ flow for 10 min, placed in an oven, and allowed to react at 70° C., typically for 3 hours. After polymerization, the crude product was reprecipitated in pentane for three times, redissolved in THF, and transferred to a clean vial. The solvent was removed by evaporation under reduced pressure. The purified polymer was then dried in vacuo overnight.

P(MMA-r-EHMA)-b-p (PEGMA-r-SPMA) synthesis OEGMA (1027.41 mg, 2.05 mmol), 3-SPMA (101.29 mg, 0.41 mmol), p (MMA-r-EHMA)₅₃ macroRAFT agent (335.743 mg, 0.0476 mmol, Mn=7056 g/mol), AIBN (0.781 mg, 0.00476 mmol), DMF (1 ml) were added and mixed in a 20 ml glass vial. The reaction mixture was deoxygenated by N₂ flow for 10 min, placed in an oven, and allowed to react at 70° C. for ~3 hours. After polymerization, the crude product was reprecipitated in pentane 3 times, redissolved in THF, and dialyzed against a 12:1 mixture of THF and water for 3 days. The solvent was removed by evaporation under reduced pressure. The purified polymer was then subject to lyophilization.

### Polymer Characterization

Molecular weight distribution curves, number-average (Mn) and weight-average (Mw) molar mass and dispersity (=Mw/Mn) of copolymers were measured by gel permeation chromatography (GPC) using an Agilent 1260 Infinity series instrument outfitted with 2 Agilent PolyPore columns (300×7.5 mm). DMF with 0.05 M LiBr was used as the eluent at 0.7 mL/min at 50° C. The columns were calibrated against Poly(ethylene glycol) standards or by light scattering. Analyte samples at 2 mg/mL were filtered through 0.2 μm polytetrafluoroethylene (PTFE) membranes (VWR) before injection (20 μL). 1H NMR spectra were recorded in DMSO-d₆ and D₂O on a Bruker Avance 400 spectrometer (400 MHZ) using a 5 mm Z-gradient BBO probe or on a Bruker Avance AV 600 spectrometer (600 MHZ) using a Z-gradient Triple Broad Band Inverse detection probe.

### Cell-Free Protein Synthesis

Cell-free protein synthesis was carried out according to PURExpress manual with modifications. In a 25 μl reaction, RHP of desired amount was added to the ribosome solution, and incubated on ice for 30 min. The polymer/ribosome samples were mixed with solution A and B containing all other required components including enzymes, RNAs, energy and nutrient molecules. 20 units RNase inhibitor and 200 ng plasmids for AqpZ-eGFP, PepTso-eGFP, and OmpT-eGFP were added, and the mixtures were incubated at 37° C. for 4 hours to complete membrane protein synthesis. The resulting products were stored at −20° C.

### Kinetics of Protein Synthesis and Western Blot Analysis.

Kinetics of cell-free synthesis were monitored by measuring the fluorescence intensity of the c-terminal GFP tag on a Tecan I-control infinite 200 plate reader. Cell-free protein synthesis mixtures were incubated in a 384-well plate at 37° C., and the GFP fluorescence (Ex/Em 488 nm/520 nm) was recorded over 4 hours. For the western blot, proteins in the cell-free protein synthesis samples were first separated by sodium dodecyl sulfate polyacrylamide gel electrophoresis (SDS-PAGE). Protein mass standards were used (Spectra multicolor broad range protein ladder, (ThermoFisher)). The proteins were then transferred to a polyvinylidene difluoride (PVDF) membrane. Multicolor protein mass standards were visible on the membrane after successful transfer. The membrane was then blocked with 3% BSA, incubated with 1:2000 mouse anti-GFP antibody, and washed. Following incubation with 1:4000 alkaline phosphatase conjugated goat anti-mouse secondary antibody, band colors were developed by using 5-bromo-4-chloro-3-indolyl phosphate (BCIP) and nitroblue tetrazolium (NBT).

Protein yield determination The protein yield of cell-free synthesis was determined by measuring the 35S-Methionine incorporated samples in a scintillation counter. EasyTag™ 35S-Methionine (7.2 μmol) was added in the 24 μl of cell-free synthesis solution, and the mixtures were incubated at 37° C. for 4 hours to incorporate 35S-Methionine into the proteins. 8 μL of the labeled cell-free reaction was mixed with 100 μL of 1M NaOH and incubated at RT for 10 min. 800 μL cold TCA/CAA mix (25% trichloroacetic acid/2% casamino acids) was added to the sample. The sample was briefly vortexed, then incubated on ice for 5 min. The precipitated proteins were collected on a paper filter by vacuum filtration and measured in a scintillation counter.

### Hydrolytic Activity Assay for Proteinase K

The hydrolytic activity of Proteinase K with different RHPs was measured by monitoring the absorbance at 410 nm to detect p-nitroaniline, which is released after the hydrolysis of 4-nitrophenyl butyrate (4-NPB). For the thermal denaturation assay, a mixture solution containing 0.07 mg/ml Proteinase K and as required, 2 mg/ml RHP in sodium phosphate buffer (50 mM, pH=7.0) was incubated at elevated temperature for 30 min. The sample containing 10 μL of the aforementioned solution, 1 μL of 4-NPB (50 mM in methanol), and 239 μL Tris buffer (50 mM, pH=8.0) was analyzed in absorption at 410 nm.

### Cell Culture

The NIH3T3 cell line was received frozen. The cells were thawed, diluted in DMEM (Gibco, 4.5 g/L glucose, L-glutamine, sodium pyruvate, 10% FBS) and incubated at 37° C. and 5% CO2. NIH3T3 cell line was divided every 3 days.

### MTT Assay

RHP was added to FBS at concentrations of 0.25, 0.5, 1, 2.5 mg/mL. The mixed solution was incubated at 52° C. for 2.5 hours. The supernatant was collected after centrifugation (12000 g, 10 min) and diluted by a factor of 5 in DMEM (Gibco, 4.5 g/L glucose, L-glutamine, sodium pyruvate), denoted as SolA. NIH3T3 cells were seeded in 96-well plates at 10000 cells/well (culture volume 100 μL/well) and incubated at 37° C. and 5% CO₂ for 16 hours. The cells were then fed with SolA and incubated at 37° C. and 5% CO₂ for 24 hours. The cell culture media was removed and 50 L MTT reagent solution (0.5 mM in DMEM, Gibco, phenol red free, 4.5 g/L glucose, L-glutamine, sodium pyruvate) was added per well. The plate was incubated at 37° C. and 5% CO₂ for 30 min. 150 μL DMSO was added per well and the plate was shaken to dissolve formazan completely. The absorbance at 570 nm was recorded using Infinite M200 microplate reader (Tecan).

### Differential Scanning Calorimetry (DSC)

DSC measurements were implemented using the MicroCal VP-DSC system (Malvern Panalytical, UK). All samples were prepared in 50 mM Sodium Phosphate buffer (pH=7.0) with 0.28 mg/mL Proteinase K and as required, 1.5 mg/mL RHP. The samples were degassed for 8 minutes using the MicroCal ThermoVac (Malvern Panalytical, UK) prior to insertion into the sample cell. When the pressure in the sample holder had stabilized at approximately 28 psi, the cells were cooled to 10° C., followed by a 15 min wait time. The thermograms were produced by measuring the difference in heat capacity between the sample solution and the reference buffer solution as the temperature was increased from 10° C. to 90° C. at a rate of 1° C./min. Following the retrieval of the thermograms, the buffer-buffer reference thermograms were subtracted using OriginLab software, as was any linear baseline observed.

### Dynamic Light Scattering (DLS)

DLS measurements were conducted on a Brookhaven BI-200SM Light Scattering System at a 90° scattering angle. The concentration for each measurement was 5 mg/ml of RHP or and 0.5 mg/ml of DHP.

### In-Situ Tryptophan Fluorescence

The thermal denaturation of the Proteinase K was measured using a LS 55 Jasco spectrofluorometer. The tryptophan residues were excited with 278 nm light and emission spectrum from 290 to 450 nm was monitored at a scan speed of 100 nm/min. The emission spectrum was recorded every 100 s at given temperature.

### Diffusion-Limited Collision Rate Estimation

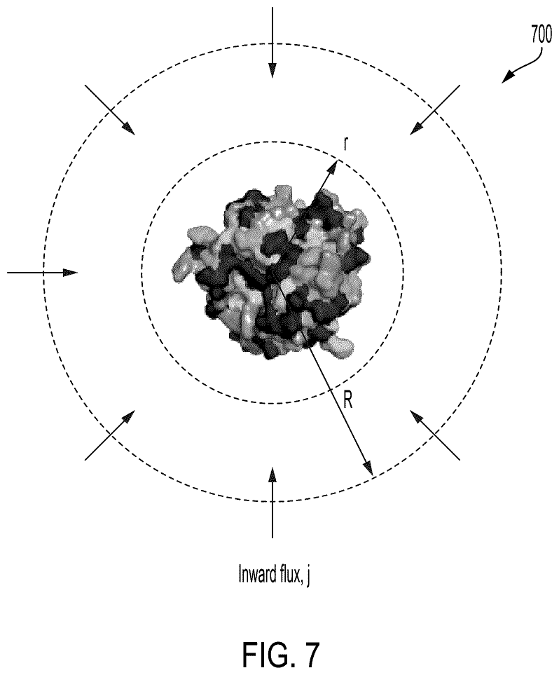

FIG. 7 illustrates a model 700 of the diffusion-limited collision rate estimation for the equations below. In one embodiment, the rate estimation may be represented by Equation (1) below:

$$\frac{d n}{dt} = j ( r ) 4 π {r}^{2} = 4 π D r {c}_{∞} = {k}_{o {n}^{{C}_{∞}}} \qquad Equation ( 1 )$$

Since proteins are around 4 nm in diameter,

$${k}_{o n} = 4 π D r = 4 π \times 50 \frac{{μm}^{2}}{s} \times 2 \times 1 {0}^{- 3} μm \times 6.02 \times 1 {0}^{23} \frac{molecules}{mol} \times \frac{1 L}{1 {0}^{15} {μm}^{3}} \approx {10}^{9} {s}^{- 1} {M}^{- 1}$$

At 2 mg/ml of RHP4 (Mn=36 kDa),

$$\frac{d n}{d t} = {k}_{o {n}^{{C}_{∞}}} = 1 {0}^{9} {s}^{- 1} {M}^{- 1} \times \frac{2 g / L}{3.6 \times 1 {0}^{4} g / mol} \approx 1 {0}^{5} {s}^{- 1}$$

### Critical Overlap Concentration of RHP4

$${c}^{*} ≅ 3 M / 4 π {N}_{A} {R}_{g}^{3} ≅ 19 wt \%$$

### Small Angle X-Ray Scattering (SAXS)

SAXS was carried out at beamline 8-ID-E at the Advanced Photon Source, Argonne National Laboratory. Samples were dissolved in water at a range of concentrations, from 0.2 to 2 wt %. Samples were measured in 2 mm boron-rich thin-walled capillary tubes and subject to multiple short exposures (5 s for each time). 2D scattering results were azimuthally averaged to produce 1D SAXS profiles. Superimposable profiles were averaged and then subtracted from the background data. The radius of gyration (Rg) of an RHP was obtained from the Guinier plot by fitting the scattering to the following Equation (2) below:

$$ln ( I ( q ) ) = ln ( {I}_{0} ) - ( {R}_{g}^{2} / 3 ) {q}^{2} \qquad Equation ( 2 )$$

### Molecular Dynamics (MD) Simulation

The final frame from an equilibrated sequence (Sequence 6) used in prior work was extracted and solvated in a system containing a pre-equilibrated hexane bubble, 4 potassium counterions, and 44,996 water molecules. The bubble is composed of 1,503 hexane molecules and was formed with 63,898 water molecules through 2 ns of equilibration and 40 ns of production simulation. The combined system was equilibrated for 2 ns and then run at production set points for 100 ns. All simulations followed the methods and parameters used in prior work, adopting the monomer parameterization methods for hexane molecules. VMD was used for visualization of the resulting trajectories. Solvent accessible surface area (SASA) calculations were performed with Amber19's LCPO default parameters. SASA for the hexane phase is taken as the SASA for the trajectory stripped of all non-polymer atoms (total RHP SASA) minus the SASA for the as-is trajectory (water accessible RHP SASA).

### Synthesis of Oligonucleotide-RHP Conjugate

Buffer A (Storage buffer): 20 mM sodium phosphate, pH=7.83
Buffer B: 200 mM sodium phosphate, 300 mM NaCl, pH=7.24
Buffer C: 100 mM sodium phosphate, 1500 mM NaCl, pH=7.24
DNA1: 1 mM in buffer A
DNA2: 1 mM in buffer A

Synthesis of RHP with dual end-functional groups of —SH and —N₃ An azido-modified chain transfer agent (2-(Dodecylthiocarbonothioylthio)-2-methylpropionic acid 3-azido-1-propanol ester) was used to synthesize RHP (RHP-N₃). Before conjugation, 1 μL of hydrazine was added into 200 μL of RHP solution (20.30 mg, in THF). The mixture was placed on a shaker for 0.5 h. RHP was purified by an Amicon-3K ultrafilter (3,000 MWCO) using DI water 6 times.

Synthesis of DNA1-RHP conjugate via thiol-maleimide addition 1.5 mg of sulfo-SMCC was dissolved in 100 μL of H₂O (heated up to 50° C. for clear solution), followed by the addition of 100 μL of buffer B. Then 10 μL of 3′-amine-modified DNA1 was added. The solution was placed on a shaker for 2 h at room temperature. The excess sulfo-SMCC was removed by an Amicon-3K ultrafilter (3,000 MWCO) using buffer B for 6 times. 5 nmol of purified sulfo-SMCC-DNA1 (90 μL in buffer C) was mixed with ~2 mg of RHP(10 μL in DMF). The reaction was placed on a shaker overnight at room temperature. After the reaction, the excess oligonucleotide was removed by Amicon-3K through washing with DI water 6 times.

Synthesis of DNA1-RHP-DNA2 conjugate via azide-alkyne cycloaddition 10 μL of 5′-hexynyl-modified DNA2 (1 mM in buffer A) was mixed with 4 μL of DNA1-RHP conjugate (~1 nmol), followed by the addition of 17 μL of DMF. 10 μL of fresh click-reaction solution (10 mM CuSO4, 50 mM TBTA, DMF/H2O=1:1) and 3 μL of sodium ascorbate (100 mM in water) was added. The mixture was placed on a shaker overnight at room temperature. The excess oligonucleotide was removed by Amicon-3K through washing with water 6 times.

### Single-Molecular Force Spectroscopy

Single-molecule force-extension measurements were carried out in a dual-trap optical tweezers instrument equipped with a 1064 nm trapping laser at a trap stiffness of 0.1 to 0.2 pN/nm. First, biotinylated double-stranded (3 kb) DNA handles with 24nt 5′ or 3′ terminal single-stranded overhangs complementary to DNA1 or DNA2, respectively, were deposited on streptavidin-coated polystyrene beads (1 μm). Individual RHP chains were captured between trapped beads by hybridization of their conjugated DNA to the overhangs in buffer containing 20 mM Tris. HCl (pH=7.2), 100 mM KCl, 10 mM MgCl2, and 5 mM BME. Pulling and relaxing force ramps were performed at a rate of 100 nm/s from less than 1 pN to up to 25 pN and repeated multiple times for each molecule. Force-extension data were collected at 667 Hz. For molecules that showed a rip in their force extension curve, passive-mode (constant trap position) measurement was also performed. Unfolding work was calculated by integrating each force-extension curve and subtracting the contribution of the worm-like-chain behavior of bare 6 kb handle DNA and unfolded RHP.

### RHP Sequence Generation

An in-house sequence simulator called Compositional Drift was used to generate 15,000 chains for each RHP ensemble (DP=50).

### Training Dataset

To train the sequence autoencoder, 30,000 membrane protein sequences and 30,000 globular protein sequences with 50% identity threshold were collected from the UniProt database. Sequences with uncommon amino acids were discarded. Each protein was reduced into four monomer codes. The assignment of each residue to its monomer equivalent is listed in Tables 1-3. For each protein sequence, a set of consecutive protein motifs were collected by moving a 50-residue long window over each sequence with 15-residue long step size, resulting in a total of 1,046,845 training sequence motifs.

### Latent Variable Model

An in-house latent variable model was developed to perform sequence dimensionality reduction and to learn protein/RHP sequence representations. The model was implemented based on a typical autoencoder (AE) architecture with an additional regression module to force the latent space to learn sequence hydrophilic-lipophilic balance (HLB) distributions. Each protein sequence motif (in its RHP equivalent form) was first one-hot encoded and then passed through the encoder. The encoder embedded each sequence into a 16-dimensional latent vector, z, which was then fed into two parallel branches: the decoder and the regressor. The decoder intended to reconstruct the input sequence from z, while the regressor predicted the HLB values of an input sequence from z. The loss function was designed to be a weighted sum of the departure (e.g., cross entropy) of the original and reconstructed sequence and the mean squared error of predicted and true HLB values. By optimizing the reconstruction and regression loss together, a more meaningful low-dimensional latent space that captures both sequential features and HLB distributions can be obtained.

Both the encoder and the decoder were implemented with simple multilayer perceptrons (MLP). Each had three fully connected layers with 256, 128, and 64 hidden units. The regression module had two fully connected layers with 16 hidden units. ReLU non-linear functions were used throughout the network, except that Sigmoid activation was used in the output layer of the decoder. The model was trained using ADAM optimizer with a learning rate of 0.001. A learning rate scheduler was used to reduce it when validation loss stopped improving. All model hyperparameters were optimized with Weights and Biases. An LSTM-based autoencoder was implemented, which demonstrated similar performance as the simpler AE variant model.

### Coacervation

The lyophilized RHP was dissolved in Mill-Q water (30 mg/ml). 1 μL of RHP solution was mixed with 29 μl sodium phosphate buffer (50 mM, pH 7.0). The coacervation/aggregation was triggered by incubating the solution at 47° C. The milky color appeared less than one minute and coacervation was confirmed by bright-field microscopy. For ssDNA partitioning, 1 mg/ml of RHP along with 1 μM ssDNA (50 mM sodium phosphate buffer, pH 7.0) were incubated at 37° C. for 10 min before imaging. Prior to dsDNA partitioning, two complementary ssDNA (5 μM for each) were mixed and heated at 95° C. for 2 min, then immediately cooled at 4° C. for 5 min. The resulting dsDNA solution was diluted to 1 μM and mixed with 1 mg/ml of RHP. The solution was incubated at 37° C. for 10 min before imaging.

### Microscopy

The differential interference contrast (DIC) microscopy was performed using a Zeiss Axiolmager M2 microscope equipped with a Zeiss Plan-Neofluar Ph3 100×/1.30 oil-immersion objective and QImaging Retiga 1350EX 1.4MP Monochrome CCD Microscope Camera. The DNA partitioning, RHP droplet fusion, and FRAP was imaged using a Zeiss LSM 880 FCS confocal microscope with X-Cite 120LED illumination system and GaAsP photon counting detector. For ssDNA partitioning, illumination was provided by a laser with the wavelength of 514 nm (for Cy3 or Cy3-Cy5 FRET pair) or 633 nm (for Cy5). Images were averaged from 8 consecutive images and were of the format 1024×10²⁴ pixels at an 8-bit depth. RHP droplet fusion was imaged every 2 seconds under transmitted light in a format of 512×512 pixels at an 8-bit depth.

Fluorescence Recover after Photobleaching (FRAP

50 μM of Cy3 amine (Lumiprobe, 410C0) was used to probe FRAP within the RHP droplets. The pinhole size was set to 1.5 micrometer section. FRAP measurement was performed by selecting a thin square region of interest (ROI) in the center of a droplet, bleaching with the 488 nm laser line at 4% for 20 s. The photobleached droplet was imaged every 5s in a format of 512×512 pixels at a 12-bit depth (4 repetitions). The FRAP analysis was performed using Fiji software. Before photobleaching, the fluorescence intensity from assigned bleached area and unbleached area was denoted as

$${I}_{b}^{before} and {I}_{u}^{before} ,$$

respectively.

$${I}_{b}^{after} ( t ) and {I}_{u}^{after} ( t )$$

represented the fluorescence intensity from bleach and unbleached area at time t after photobleaching. All fluorescence intensities were normalized by the area. The center-to-ratio was denoted as

$${r}^{before} = \frac{{I}_{b}^{before}}{{I}_{u}^{before}} , {r}^{after} ( t ) = \frac{{I}_{b}^{after} ( t )}{{I}_{u}^{after} ( t )} .$$

Thus, the recovery rate (R) was calculated by

$$R = \frac{{r}^{after} ( t ) - {r}^{after} ( 0 )}{{r}^{before} - {r}^{after} ( 0 )}$$

The recovery rate curve was fitted by an exponential function:

R=A(1−e^{t/t}),

where A and τ represent the maximal recovery rate and recovery half time, respectively.

The present disclosure also provides a hybrid variational autoencoder for designing random heteropolymers as protein mimics. Synthetic random heteropolymers (RHPs), consisting of a predefined set of monomers, offer an approach toward the design of protein-like materials. These RHPs, if designed appropriately, can mimic protein behavior and function. As such, there is a need for computational tools to efficiently guide RHP design. This gap is bridged by developing DeepRHP, a modified variational autoencoder (VAE) model under a semi-supervised framework. By equipping a classical VAE with an additional feature-based VAE, DeepRHP forces the latent space to capture structures of critical chemical features as well as individual RHP sequence patterns. In this sense, the method of the present disclosure is versatile by allowing any relevant features to be incorporated in a hybrid manner. The effectiveness of DeepRHP is demonstrated by suggesting potential monomer compositions that stabilize membrane proteins (e.g. Aquaporin Z) in non-native environments and cross-validating the prediction with published results. The concordance between the model of the present disclosure and true RHP function suggests strong potential in utilizing hybrid autoencoder architectures to guide RHP design for proteins and other biological compounds.

There is a significant interest in engineering synthetic materials capable of replicating protein functions while satisfying stability and compatibility with device fabrication and integration. However, it remains an insurmountable challenge to synthesize sequence-specific polymers. This has led to a recent surge of research in designing protein-like random heteropolymers. Random heteropolymers (RHPs) are an ensemble of many polymer chains with each being composed of monomers arranged in random order. Recent developments have demonstrated that RHPs can act as chaperone proteins for protein stabilization in nonbiological environments, a critical bottleneck to fabricate protein-embedded plastics for end-of-life plastic degradation. In addition, RHPs can be designed to act as channel proteins for rapid and selective proton transportation, important for fuel cells and energy storage.

Despite the fact that RHPs can serve as great biofunctional materials, designing RHPs with desired function is challenging because both the exact monomeric sequences and conformations of synthetic RHP chains are not deterministic. Traditional protein design methods rely heavily on high-throughput sequencing data and 3D structures. For example, directed evolution methods evolve protein function by iteratively mutating a selected protein sequence, while de novo methods build novel proteins that fold into a certain structure. Without exact sequences and structures, there are no rational design principles for creating suitably functional RHP chains. Current RHP designs are largely empirical and depend on time-intensive lab screenings over various monomer compositions and chain lengths. For each RHP made in the lab, ensembles of thousands of sequences are simulated under the same monomer composition in order to understand why certain compositions perform better than others. In this process, scientists face two practical design questions that can potentially accelerate progress if answered: 1) How many monomers should be included in a RHP system? Recent results show that RHPs can mimic protein function with only four monomers, but it remains unclear how many monomers are enough to include in the alphabet. 2) How can one find monomer compositions corresponding to specific protein functions?

Answering these questions requires new methods to model and analyze RHP sequences as an ensemble instead of as individual chains. There is very limited literature on computational methods of modeling RHPs. As the only two examples used Hidden Markov Models to characterize the functionality of proton-transporting RHPs and utilized Gaussian process regression coupled with Bayesian optimization for optimal copolymer identification.

Here, DeepRHP is proposed. DeepRHP is a modified variational autoencoder trained in a semi-supervised manner, for modeling general RHP sequence data and discovering RHP compositions for protein function. This tool serves as a first step that can guide RHP design by examining their protein-mimicking behavior. The key contributions of this study are: To answer RHP design questions with deep learning. DeepRHP learns interpretable latent representations for RHP sequences and provides a platform to perform similarity analysis between target proteins and RHP sequences in an ensemble. DeepRHP provides insights into the two important design parameters: monomer alphabet size and monomer composition. Show that the best monomer composition suggested by DeepRHP matches published experimental results. DeepRHP is flexible enough to incorporate any function-related chemical features for a wide variety of protein functions.

VAE-based architectures are some of the first model classes used to identify latent representations for biological sequences, and are useful in downstream tasks like identifying mutation effects and designing novel functional proteins. Therefore, the same machine learning theory in macromolecular cheminformatics can be leveraged by the present disclosure, specifically in this instance of using RHPs to mimic natural biopolymers.

### Data

This system of the present disclosure consists of four methacrylate-based monomers: methyl methacrylate (MMA), 2-ethylhexyl methacrylate (EHMA), oligo (ethylene glycol) methacrylate (OEGMA), and 3sulfopropyl methacrylate potassium salt (SPMA). MMA and EHMA are the hydrophobic monomers used to tailor overall hydrophobicity, while OEGMA and SPMA are the hydrophilic monomers used to reduce the aggregation propensity of RHPs.

Compositional Drift, a software developed to simulate 10,000 sequences per monomer composition listed in Table 4, is used. This software uses established mathematical copolymer models in tandem with Monte-Carlo simulation to calculate RHP sequences based on experimental conditions. It was shown that, while each chain simulated is random at the sequence level, each RHP chain contains characteristic segments that have a well-defined statistical distribution. The reasoning behind the monomeric compositions for each specific RHP is further discussed below.

Also 30,000 membrane protein sequences and 30,000 globular protein sequences with 50% identity threshold were collected from the UniProt database. Some common pre-processing procedures were performed, including discarding sequences with uncommon amino acids and lengths. Each protein was then reduced into its monomer-equivalent form according to the assignment in Table 5 below. Note that the reduction of protein alphabet is not uncommon in protein sequence analysis. Here the reduction rule is based on monomer hydrophobicity and charge.

### DeepRHP Methodology

In order to address the domain questions raised above, DeepRHP, a modified variational autoencoder, was developed. An example is provided in Tables 5 and 6 below:

TABLE 5
Two and four-monomer composition of RHPs used for training

| | RHP | MMA | OEGMA | EHMA | SPMA |
|---|---|---|---|---|---|
| 2 | A | 0 | 10 | 90 | 0 |
| Mon. | B | 0 | 30 | 70 | 0 |
| | C | 0 | 50 | 50 | 0 |
| | D | 0 | 70 | 30 | 0 |
| | E | 0 | 90 | 10 | 0 |
| 4 | 1 | 70 | 25 | 0 | 5 |
| Mon. | 2 | 65 | 25 | 5 | 5 |
| | 3 | 60 | 25 | 10 | 5 |
| | 4 | 50 | 25 | 20 | 5 |
| | 5 | 40 | 25 | 30 | 5 |
| | 6 | 20 | 25 | 50 | 5 |
| | 7 | 0 | 25 | 70 | 5 |

TABLE 6
Amino acid (protein) to monomer (RHP) conversion

| | Monomer | |
|---|---|---|
| Amino acid | equiv. | Property |
| C, Y, A, T, G | MMA | Hydrophobic |
| S, Q, H, N, P | OEGMA | Hydrophilic |
| L, I, F, W, V, M | EHMA | Very |
| | | Hydrophobic |
| E, D, R, K | SPMA | Charged |

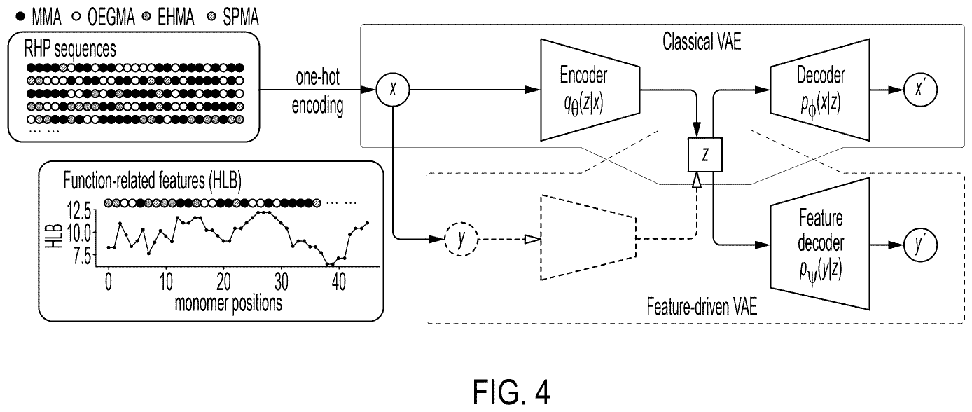

Under semi-supervised framework for learning low dimensional RHP sequence representations. The model architecture is illustrated in FIG. 4. It is assumed that the sequence family X follows a probability distribution p (x) and the existence of an underlying latent variable z~N (μ_{z}, Σ_{z}) that captures intrinsic unobserved sequence properties. For each sequence x, there also exists a function-related feature y, which can be considered as a deterministic transformation of x. In the application case presented below, y is the average hydrophilic-lipophilic balance (HLB) value of sliding windows along each sequence. HLB measures local hydrophobicity and solubility distributions and is closely related to RHP functions. The other function-related chemical features (e.g. HLB) can be introduced to guide the formation of the latent space.

To incorporate a chemical feature y into the VAE model, a feature-driven VAE is added in parallel with the classical VAE. y and x share the common latent variable z. This is equivalent to simultaneously training two VAEs with shared latent embeddings, and the encoder relies only on x since y is a direct transformation of x, as indicated by the dashed lines in FIG. 4.

The objective is still to maximize the log-likelihood log p (x) given sequence data X as shown in Equation (3):

Equation (3): logp(x)=logp(x|z)p(z)dz

Under the regular VAE setting, Equation 1 can be bound by the well-known evidence lower bound (ELBO) defined by Equation (4):

Equation (4): logp(x)≥E_{q}[logp(x|z)]−D_{KL}(q(z|x)∥p(z))

where q is the learned posterior of the normal distribution family. In practice, p and q are learned by the encoder and decoder and their weights are optimized through gradient descent.

Traditionally, the reconstruction loss term is approximated by mean-squared error for continuous input, or cross entropy loss for discrete input. By imposing this hybrid architecture, the reconstruction loss can be approximated through both the classical VAE on x, the feature-driven VAE on y, or a weighted sum of both. The modified ELBO that considers both sequence structures and chemical features is then formulated as Equation (5):

Equation (5): logp(x)≥αE_{q}[logp(x|z)]+(1−α)E_{q}[logp(y|z)]−Dₖₗ(q(z|x)∥p(z))

where α is a hyperparameter that dictates how much weight is placed on each approximation term. In the present case, the first two terms of Equation 4 are approximated as follows in Equations (6) and (7):

Equation (6): E_{q}[log p(x|z)]~ΣₓΣₗp(xₗ)logp(xₗ|z)

$${E}_{q} [ log p ( y \vert z ) ] \approx - {\sum }_{y} {‖ y - {y}^{′} ‖}_{2}^{2} , \qquad Equation ( 7 )$$

where y is the output of feature-based decoder denoted by the blue shading in FIG. 4.

By optimizing the reconstruction loss in this hybrid manner, a meaningful low-dimensional latent space is obtained that captures the sequence structure relevant to the desired protein function. Additionally, the method of the present disclosure comes with interpretability benefits that classical VAEs often lack. Existing works usually concatenate all features together into a single vector for the encoder. The resulting latent space is then obscured, as no physical meanings can be derived for the principal directions. In contrast, the hybrid training of the present disclosure leads to meaningful visualizations of the data because the latent variables are directly linked to the chemical features.

Both the encoder and the decoder were implemented with multilayer perceptrons using PyTorch. Each has three fully connected layers with 256, 128, and 64 hidden units, respectively. The feature decoder has two fully connected layers with 32 hidden units. ReLU activation functions were used as non-linearities throughout the network, except in the output layer of the decoder where Sigmoid activation was used instead. The model was trained using the ADAM optimizer with a learning rate of 0.0001. A learning rate scheduler was used when validation loss stopped improving.

### Results and Discussion

Aquaporins (Aqp) are membrane channel proteins that facilitate water transport between cells. Membrane proteins are unstable and prone to aggregation even under mild experimental conditions. Previous works successfully stabilized Aquaporin Z (AqpZ) and preserved its function in non-native environments with the presence of RHPs. The present disclosure demonstrates how DeepRHP can be used to accelerate RHP design by identifying promising monomer compositions.

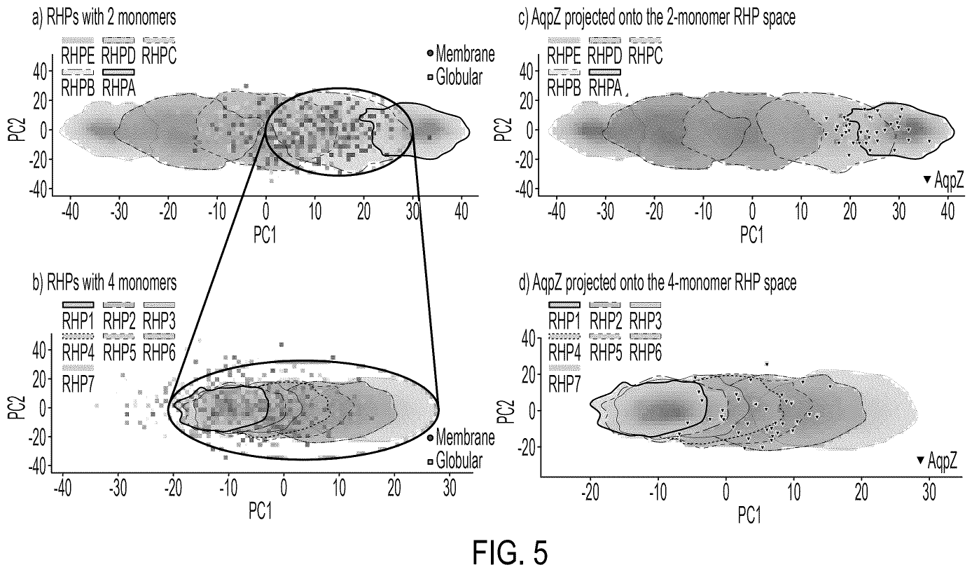

Previous works chose to use 70% hydrophobic monomers and 30% hydrophilic monomers in their RHP system based on a crude protein surface analysis on four protein sequences. First, this distribution of monomer hydrophobicities was validated. The latent factors of the two monomer RHPs and natural proteins are projected onto a two-dimensional space using Principal Component Analysis (PCA), as shown in graph a) of FIG. 5. All two-monomer RHPs are composed of one hydrophobic monomer (EHMA) and one hydrophilic monomer (OEGMA). The compositions of RHP A through RHP E listed in Table 5 are selected to sufficiently reflect this hydrophobicity range. It is observed that PC1 correlates with hydrophobicity as RHPs span left to right, with left being least hydrophobic to right being most hydrophobic. The majority of membrane and globular proteins overlap with RHP B and RHP C, suggesting these two RHP compositions are most similar to natural proteins. On the other hand, most hydrophobic membrane proteins overlap with RHP B (30% hydrophilic, 70% hydrophobic), confirming that 30:70 is a good balance for the two-monomer system.

The performance of the 30:70 distribution of hydrophilic and hydrophobic monomers is then fine-tuned by increasing the number of monomers from two to four as shown in graph b) of FIG. 5. A library of four-monomer-based RHPs was designed by varying the MMA: EHMA ratio. The specific monomer composition is shown in Table 5. Each of RHP 1 through RHP 7 is still composed of 30% hydrophilic monomers (OEGMA+SPMA) and 70% hydrophobic monomers (MMA+EHMA).

Previous works did not rationalize the choice of four monomers for their design of protein-like RHPs. The approach of the present disclosure explains why the two-monomer alphabet size is insufficient. In graph b) of FIG. 5, each of the RHP ensembles can be considered as a subset of RHP B and occupies a much more localized natural protein sequence space with smaller variance.

In graphs c) and d) of FIG. 5, AqpZ is projected onto the two-monomer and four-monomer PCA spaces, respectively. In the two-monomer setting, the RHP B space is much larger than the span of AqpZ. In the four-monomer setting, however, the AqpZ projections cover the RHP 4 and RHP5 spaces almost entirely. Therefore, it is believed that the two-monomer sequence space is too broad with respect to proteins while the four-monomer sequence space is more localized, offering stability in synthesizing RHPs.

In addition to providing heuristics regarding the number of monomers, DeepRHP sheds light on the choice of monomer compositions. In graph d) of FIG. 5, there is a large overlap between the projected proteins and the RHP 4 and RHP 5 contours. Wet-lab experiments demonstrated that the optimal RHP has the same monomer ratio as that of RHP 4 and is capable of stabilizing AqpZ. Thus, the overlap between RHPs and AqpZ in the PCA space can modulate their sequence correlation and molecular interactions in the aqueous solution. This indicates that the latent embeddings discovered by DeepRHP are chemically meaningful and play a key role in discovering RHPs that provide strong performance.

Thus, in the present disclosure, DeepRHP, a hybrid variational autoencoder model, is developed to guide RHP design. The model of the present disclosure suggests the feasibility of four-monomer compositions to stabilize ApqZ, matching the respective wet-lab experiment. In ablation studies, the model of the present disclosure outperforms a singular classical VAE without the additional decoding regressor.

Overall, DeepRHP holds much promise for the future of integrating deep learning techniques, specifically VAEs, into RHP design. Hybrid VAE architectures like DeepRHP possess many advantages. First, they are flexible and can be trained on any sequence family with variable sequence lengths and no multiple sequence alignment is needed. DeepRHP is also flexible due to its flexibility in supervision. It can be totally unsupervised when no prior knowledge on RHP subpopulations is available, or it can also be semi-supervised by combining function-related chemical features with vast amounts of sequence data to improve interpretability of latent variables.

Future work in this regime includes strengthening the quantitative assessment of DeepRHP. The model of the present disclosure is currently assessed in a qualitative manner and validated using laboratory results. DeepRHP can be improved by developing a quantitative measure to evaluate the quality of the latent representations. For instance, further downstream tasks such as classifying specific membrane proteins and evaluating similarities between each RHP and their target proteins can be completed.

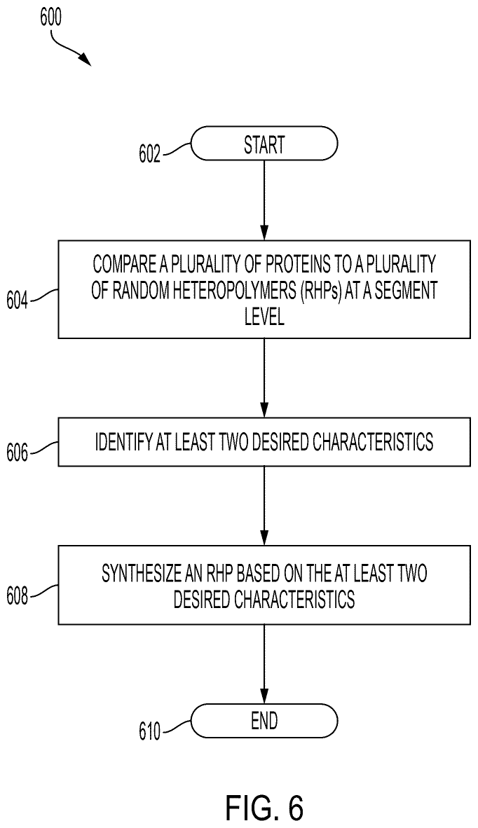

FIG. 6 illustrates a flow chart of an example method 600 for synthesizing an RHP of the present disclosure. The method 600 may be executed by a one or more processors of one or more computing devices. For example, the method 600 may be stored as instructions stored on a non-transitory computer readable medium of a computing device and executed by a processor to perform the functions described herein.

The method 600 begins at block 602. At block 604, the method 600 compares a plurality of proteins to a plurality of RHPs at a segment level. In one embodiment, the plurality of proteins may be truncated into 50-mer segments and the comparison can be performed using PCA, as described above and illustrated in FIGS. 1 and 2.

At block 606, the method 600 identifies at least two desired characteristics. The desired characteristics may include hydrophobicity and charge. The hydrophobicity may be separated into additional characteristics of hydrophilic, hydrophobic, and very hydrophobic.

At block 608, the method 600 synthesizes an RHP based on the at least two desired characteristics. For example, the RHP may be synthesized using a modified variational autoencoder, as described above and illustrated in FIG. 4. In one embodiment, the RHPs that may be synthesized may include at least two of methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA), or oligo (ethylene glycol) methacrylate (OEGMA) 300 Da or 500 Da. At block 610, the method 600 ends.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

What is claimed is:

1. A method of heteropolymer design and synthesis, comprising:
(a) comparing a plurality of proteins to a plurality of random heteropolymers (RHPs) at a segment level;
(b) identifying at least two desired characteristics; and
(c) synthesizing an RHP based on the at least two desired characteristics.

2. The method of claim 1, wherein the plurality of proteins is truncated into 50-mer segments for the comparing.

3. The method of claim 1, wherein the comparing is performed using principal component analysis (PCA).

4. The method of claim 3, wherein a first principal component correlates to hydrophobicity of the plurality of proteins.

5. The method of claim 4, wherein a second principal component correlates with a sequential arrangement of different blocks within the plurality of proteins.

6. The method of claim 1, wherein the at least two desired characteristics comprise hydrophobicity and charge.

7. The method of claim 1, wherein the plurality of random heteropolymers comprises methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA), or oligo (ethylene glycol) methacrylate (OEGMA).

8. The method of claim 7, wherein the RHP that is synthesized is a composition comprising at least two of:
methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA), or oligo (ethylene glycol) methacrylate (OEGMA).

9. The method of claim 7, wherein the RHP that is synthesized is a composition comprising a combination of (i) OEGMA, (ii) 3-SPMA or DMAEMA, (iii) MMA, and (iv) 2-EHMA.

10. The method of claim 1, wherein:
the plurality of proteins is truncated into 50-mer segments for the comparing;
the comparing is performed using principal component analysis (PCA);
a first principal component correlates to hydrophobicity of the plurality of proteins;
a second principal component correlates with a sequential arrangement of different blocks within the plurality of proteins;
the at least two desired characteristics comprise hydrophobicity and charge;
the plurality of random heteropolymers comprises methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA), or oligo (ethylene glycol) methacrylate (OEGMA); and
the RHP that is synthesized is a composition comprising methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA), or oligo (ethylene glycol) methacrylate (OEGMA).

11. The method of claim 1, wherein:
the plurality of proteins is truncated into 50-mer segments for the comparing;
the comparing is performed using principal component analysis (PCA);
a first principal component correlates to hydrophobicity of the plurality of proteins;
a second principal component correlates with a sequential arrangement of different blocks within the plurality of proteins;
the at least two desired characteristics comprise hydrophobicity and charge;
the plurality of random heteropolymers comprises methyl methacrylate (MMA), 2-ethylhexyl methacrylate (2-EHMA), 3-sulfopropyl methacrylate potassium salt (3-SPMA), 2-(dimethylamino)ethyl methacrylate (DMAEMA), or oligo (ethylene glycol) methacrylate (OEGMA); and
the RHP that is synthesized is a composition comprising at least two of: the composition comprises a combination of (i) OEGMA, (ii) 3-SPMA or DMAEMA, (iii) MMA, and (iv) 2-EHMA.

12. The method of claim 1, wherein the synthesizing is performed using a modified variational autoencoder trained with two and four monomer composition RHPs and an average hydrophilic-lipophilic balance (HLB) value of sliding windows along each sequence of the two and four monomer composition RHPs.

13. The method of claim 11, wherein the synthesizing is performed using a modified variational autoencoder trained with two and four monomer composition RHPs and an average hydrophilic-lipophilic balance (HLB) value of sliding windows along each sequence of the two and four monomer composition RHPs.

14. The method of claim 8, wherein the OEGMA comprises a molecular weight of 300 Daltons (Da) or 500 Da.

15. The method of claim 8, wherein the composition comprises a molar ratio of 30% hydrophilic monomers and 70% hydrophobic monomers.

16. The method of claim 8, wherein the composition comprises a combination of OEGMA, 3-SPMA, MMA, and 2-EHMA.

17. The method of claim 11, wherein the composition comprises a combination of OEGMA, 3-SPMA, MMA, and 2-EHMA.

18. The method of claim 8, wherein the composition comprises a molar composition of approximately 25% OEGMA, 5% 3-SPMA, 0-70% MMA, and 0-70% 2-EHMA.

19. The method of claim 8, wherein the composition comprises a combination of OEGMA, DMAEMA, MMA, and 2-EHMA.

20. The method of claim 11, wherein the composition comprises a combination of OEGMA, DMAEMA, MMA, and 2-EHMA.
